(19) United States
(12) Plant Patent Application Publication
(10) Pub. No.: US 2003/0056261 P1
(43) Pub. Date: Mar. 20, 2003

(54) Chrysanthemum plant named 'Machismo Time'

(76) Inventor: Mark Roland Boeder, J H Den Haag (NL)

Correspondence Address: HOWREY SIMON ARNOLD & WHITE, LLP, BOX 34, 301 RAVENSWOOD AVE., MENLO PARK, CA, 94025, US

(21) Appl. No.: 09/957,493

(22) Filed: Sep. 19, 2001

(51) Int. Cl. A01H 5/00

(52) U.S. Cl. PLT/286

(57) ABSTRACT

A new variety of Chrysanthemum plant named ‘Machismo Time,’ having a vigorous upright of branching habit with many yellow/red bi-coloured single flowers giving a slightly domed capitulum.

### LATIN NAMEOF THEGENUS AND SPECIES

Botanical classification:Dendranthemagrandiflora.

### VARIETY DENOMINATION

The new Chrysanthemum variety denomination is ‘Machismo Time.’

### BACKGROUND OF THE INVENTION

The present invention comprises a new and distinct cultivar of Chrysanthemum botanically known as Dendranthema grandiflora, and referred to by the cultivar name ‘Machismo Time.’

‘Machismo Time,’ identified as 20360-1 originated from a cross made by Mark Boeder in a controlled breeding program in Chichester, West Sussex, United Kingdom. The female parent of the new variety is undistributed—unpatented seedling P333C6 and the male parent is the variety known as an undistributed—unpatented seedling P333C5. The new variety ‘Machismo Time’ has been asexually reproduced by vegetative cuttings, in Chichester, West Sussex, United Kingdom and the distinguishing characteristics are retained through successive generations of asexual reproduction.

### BRIEF SUMMARY OF THE INVENTION

‘Machismo Time’ is a pot type of Chrysanthemum plant variety having a slightly domed capitulum of yellow/red bi-coloured single flowers.

### Comparison with Parent

Plants of the new Chrysanthemum variety ‘Machismo Time’ are similar to the parent cultivar P333C6 in plant habitat and growth rate. In side-by-side comparisons in Chichester, West Sussex, United Kingdom, under commercial practice, plants of the new Chrysanthemum variety ‘Machismo Time’ compared to plants of the parent cultivar P333C6 in the following characteristics.

1. The new variety ‘Machismo Time’ produces more distinctive flowers, whereas the parent cultivar P333C6 produces less demarcation in bi-coloured flowers.

2. Plants of the new variety ‘Machismo Time’ have smaller and shorter inflorescence than plants of the parent cultivar P333C6.

3. ‘Machismo time’ has less vigour than the parent cultivar P333C6, and a more branching habit.

### Comparison with Other Varieties

Plants of the new Chrysanthemum variety ‘Machismo Time’ are dissimilar to the cultivar ‘Pelee’ in plant habitat and growth rate. However, in side-by-side comparisons in Chichester, West Sussex, United Kingdom, under commercial practice, plants of the new Chrysanthemum variety ‘Machismo Time’ differed from plants of the parent cultivar ‘Pelee’ in the following characteristics.

1. The new variety ‘Machismo Time’ produces yellow/red single flowers whereas the cultivar ‘Pelee’ produces orange/yellow single flowers.

2. Plants of the new variety ‘Machismo Time’ have smaller and shorter inflorescence than plants of the parent cultivar ‘Pelee.’

3. Two day difference response time.

4. The bi-colour characteristics of ‘Pelee’ are less defined than ‘Machismo Time.’

### BRIEF DESCRIPTION OF ILLUSTRATIONS

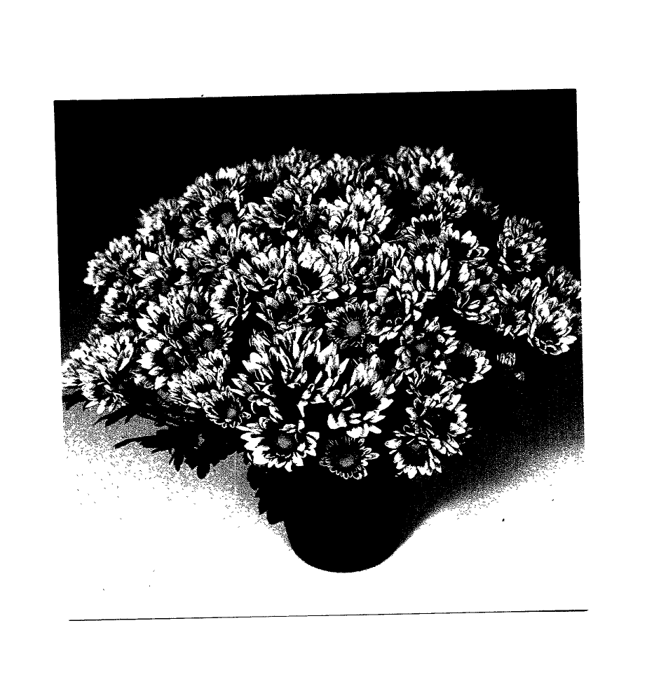

Typical specimens of the plant and flowers for the new Chrysanthemum variety ‘Machismo Time’ are shown in the accompanying photograph. The colours shown are as true as possible within the usual limits of this kind of illustration. FIG. 1 is a whole plant view of the new variety ‘Machismo Time’ grown in a pot. The plant shown in the illustration is 56 days from the start of Short Days.

### DETAILED BOTANICAL DESCRIPTION

The following description of the new Chrysanthemum variety ‘Machismo Time’ is of plants grown in a greenhouse in Chichester, West Sussex, United Kingdom in the month of August. The cultivar has not been observed under all possible environmental conditions. The phenotype may vary significantly with variations in the environment such as temperature, length of day and light intensity, without any variance in genotype. The commercial classification of the new variety is pot Chrysanthemum.

Plants of the new variety have been grown successfully under temperature conditions averaging about 18° C. at night and about 24° C. to 25° C. during the day under light conditions of about 5,000 to 6,000 foot candles. The plants respond well to the use of growth retardant, such as three B9 treatments at about 2500 ppm. To produce a commercial product the plants may be pinched once with the centre bud removed. The typical container size for commercial growth is 14 cm. It has been observed that the shelf life of the new variety is about 22 days with a response time of about 7½weeks. The new variety is suitable for a temperature range of 16° C. to 25° C.

The new variety may be produced as a spray or disbud. The following description is with respect to a plant produced as Spray pot Chrysanthemums. In the description of this new Chrysanthemum variety, colour values have been taken from The Royal Horticultural Society Colour Chart (R.H.S.C.C.).

### Plant

Plant type: Pot Chrysanthemum

Habit: Upright Branching

Height: Tall 23 to 27 cm.

Width: Near 30 cm.

Branching characteristics: Upright—Spreading.

Length of Lateral Branches: 12 to 18 cm.

Number of Breaks from Pinch: 4 to 5.

Stem Colour: 147 B.

Response Time: 52 days.

Vigour: Tall.

Shelf Life: 22 days.

Disease: No observations

Pest: No observations

Growth retardant type and treatment: B9 85% 3 applications at 2.5 gm/litre at 2 days, 21 days, 31 days after propagating unrooted vegetative cuttings. The plants were grown for two weeks in Long Day conditions (20 hours of light) and then transferred to Short Day conditions (13 hours of dark). Propagation:

Type.—Vegetative propagation via stem cuttings

Time to rooting.—10-12 days with soil temperatures of 18° C.

Rooting habit.—Fine and fibrous

Other treatments or conditions: None

### Foliage

Number of Leaves per Lateral Branch:8-11

Compound or single: Single

Arrangement of Leaves: Alternate

Shape of Leaf.—Typically 5 lobed oblong—ovate.

Size of Leaf.—Width (cm): Near 4½cm Length (cm): Near 9 cm.

Leaf Apex.—Acute/Cupsidate.

Base.—Obtuse.

Attachment.—Petioled.

Aspect.—Slightly undulating.

Margin.—Palmately Lobed.

Surface characteristics.—Top: Slightly pubescent Bottom: Slightly pubescent

Petiole: Colour: Near 143 B Length: Near 2 cm.

Venation: Net—prominent mid vein at undersides Colour. upper side: Near 144 B under side: Near 144 B

Colour: Mature Leaf, upper side: Near 143 B; under side: Near 143 B Young Leaf, upper side: Near 143 B; under side: Near 143 B

### Flower

Flower appearance: Cupped—circular

Flower type: Single (Daisy)

Flower form: Cupped

Flower shape: Circular

Flowering habit: Upright

Number of blossoms per branch: 7 to 10

Inflorescence form: Slightly domed

Depth of fully expanded blossoms.—Near 1½cm.

Diameter of fully expanded blossoms.—Near 4½cm.

Sepals:

Number.—18-25

Colour.—Near 137 B

Length.—0.8 to 1.2 cm.

Width.—0.2 to 0.3 cm.

Texture/Appearance.—Slightly pubescent

Peduncle: Peduncle length is shortest at the terminal flower and progressively longer moving down the stem; longest length is on average 12 cm, shortest length is on average 8 cm:

Length.—8 cm.

Colour.—Near 137 B.

Surface.—Slight Pubescence.

Habit.—Slightly curving.

Strength.—Medium.

Pedicel: Length: terminal: 5 to 6; lateral 9 to 10

Colour.—Near 137 B.

Surface.—Slightly pubescent.

Habit.—Slightly curving.

Strength.—Medium.

Ray Florets:

Form/Shape.—Slightly twisted.

Texture/Appearance.—Shiny.

Number per flower.—26 to 35.

Length.—1.8 to 2.0 cm.

Width.—Near 0.6 cm.

Apex.—Round.

Base.—Tapered.

Margin.—Entire.

Disc florets:

Form/Shape.—Cylindrical.

Texture/Appearance.—Shiny.

Number per flower.—150 to 200.

Length.—Near 0.4 to 0.5 cm;

Width<0.1 cm.

Diameter of disc.—1.5 cm.

Fragrance: Slight

Flower Bud (at onset of colour):

Length.—Near 0.7 cm.

Diameter.—Near 0.9 cm.

Form/shape.—Globular.

General flower colour: Golden yellow with distinctive crimson surround to disc.

1. Ray florets, upper side: Immature: Tip near 13 A Base near 44 A Mature: Tip near 9 A Base near 44 B Older/Fading: Tip near 9 B Base near 44 B

2. Ray florets, under side: Immature: Near 2 B Mature: Near 2 B Older/Fading: Tip near 9 B Base near 44 B

3. Disc florets: Immature: Near 2 B Mature: Near 2 B Older/Fading: Near 2 B

4. Bud: Near 138 B

Flower progression with age: Flowers hold forn—shape—habit and colour well during ageing.

Other Distinguishing Characteristics: None

### Reproductive Organs

Ray Florets: Pistillate

Pistil number.—26 to 35.

Stigma colour.—Near 6 C.

Stigma shape.—Forked.

Style Colour.—Near 6 C.

Style Length.—Near 0.2 cm.

Disc Florets: Pistillate

Pistil number.—7 100 when used as a parent (female)

Stigma colour.—Near 6 C.

Stigma shape.—Forked.

Style Colour.—Near 6 C.

Style Length.—Near 0.3 cm.

Staninate.—

Stamen Number.—<150.

Anther Shape.—Cylindrical.

Anther Colour.—Near 7 C.

Pollen Colour.—Near 14 A.

Pollen Quantity.—Medium.

What is claimed is:

1. A new and distinct variety of Chrysanthemum plant, substantially as described and illustrated herein.
